(19) United States
(12) Patent Application Publication
(10) Pub. No.: US 2007/0254362 A1
(43) Pub. Date: Nov. 1, 2007

(54) COMPOSITIONS AND METHODS EMPLOYING UNIVERSAL-BINDING NUCLEOTIDES FOR TARGETING MULTIPLE GENE VARIANTS WITH A SINGLE siRNA DUPLEX

(75) Inventors: Steven C. Quay, Seattle, WA (US); James Anthony McSwiggen, Bothell, WA (US)

Correspondence Address: NASTECH PHARMACEUTICAL COMPANY INC, 3830 MONTE VILLA PARKWAY, BOTHELL, WA, 98021-7266, US

(73) Assignee: Nastech Pharmaceutical Company Inc.

(21) Appl. No.: 11/610,403

(22) Filed: Dec. 13, 2006

Related U.S. Application Data
(60) Provisional application No. 60/796,274, filed on Apr. 27, 2006.

(51) Int. Cl. C12N 5/06; C07H 21/02

(52) U.S. Cl. 435/375; 536/24.5

(57) ABSTRACT

Provided are siRNA molecules of between about 15 base-pairs and about 40 base-pairs comprising one or more universal-binding nucleotide such as inosine, 1-β-D-ribofuranosyl-5-nitroindole, and 1-β-D-ribofuranosyl-3-nitropyrrole, compositions comprising one or more universal-binding nucleotide comprising siRNA, and methods for making and for using such universal-binding nucleotide comprising siRNA molecules to increase the specific binding of the modified siRNA molecule to variants of a target sequence such as, for example, when in contact with a biological sample and to reduce off-target effects of the siRNA molecule.

This patent application claims priority under 35 U.S. § 119(e) of U.S. Provisional Application No. 60/796,274 filed Apr. 27, 2006, the contents of which are incorporated herein by reference.

### BACKGROUND OF THE DISCLOSURE

1. Technical Field of the Invention

The present invention relates to the treatment of disorders by means of RNA interference (RNAi). More specifically, the present disclosure relates to the targeted delivery of small inhibitory nucleic acid molecules (siRNA) that are capable of mediating RNAi against genes, and variants thereof, wherein the siRNA comprise one or more universal-binding nucleotide such as, for example, inosine, 1-β-D-ribofuranosyl-5-nitroindole, and 1-β-D-ribofuranosyl-3-nitropyrrole.

2. Description of the Related Art

RNA interference refers to the process of sequence-specific post-transcriptional gene silencing in animals mediated by small inhibitory nucleic acid molecules (siRNAs) a double-stranded RNA (dsRNA) that is homologous in sequence to a portion of a targeted messenger RNA. See Fire, et al., Nature 391:806, 1998, and Hamilton, et al., Science 286:950-951, 1999. These dsRNAs serve as guide sequences for the multi-component nuclease machinery within the cell that degrade the endogenous-cognate mRNAs (i.e., mRNAs that share sequence identity with the introduced dsRNA).

The process of post-transcriptional gene silencing is thought to be an evolutionarily-conserved cellular defense mechanism used to prevent the expression of foreign genes and is commonly shared by diverse flora and fauna. Fire, et al., Trends Genet. 15:358, 1999. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response through a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

RNAi has been studied in a variety of systems. Fire et al. were the first to observe RNAi in C. elegans. Nature 391:806, 1998. Bahramian & Zarbl and Wianny & Goetz describe RNAi mediated by dsRNA in mammalian systems. Molecular and Cellular Biology 19:274-283, 1999, and Nature Cell Biol. 2:70, 1999, respectively. Hammond, et al., describe RNAi in Drosophila cells transfected with dsRNA. Nature 404:293, 2000. Elbashir, et al., describe RNAi induced by introduction of duplexes of synthetic 21-nucleotide RNAs in cultured mammalian cells including human embryonic kidney and HeLa cells. Nature 411:494, 2001.

Recent work in Drosophila embryonic lysates revealed certain requirements for siRNA length, structure, chemical composition, and sequences that are essential to mediate efficient RNAi activity. Elbashir, et al., EMBO J. 20:6877, 2001. These studies demonstrated that 21-nucleotide siRNA duplexes are most active when containing 3′-terminal dinucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) are tolerated. Single mismatch sequences in the center of the siRNA duplex abolish RNAi activity.

The position of the cleavage site in the target RNA is defined by the 5′-end of the siRNA guide sequence rather than the 3′-end of the guide sequence. Elbashir, et al., EMBO J. 20:6877, 2001. Other studies indicate that a 5′-phosphate on the target-complementary strand of an siRNA duplex is required for siRNA activity and that ATP is utilized to maintain the 5′-phosphate moiety on the siRNA. Nykanen, et al., Cell 107:309, 2001.

RNA interference is emerging as a promising technology for modifying expression of specific genes and therefore is useful as a therapy for a wide range of diseases and disorders amenable to treatment by reduction of endogenous or viral gene expression (e.g., the reduction of Tumor Necrosis Factor-alpha in the treatment of rheumatoid arthritis or the reduction of viral genes in the treatment of virally induced disease such as influenza and AIDS).

The mechanism by which dsRNA duplexes mediate targeted gene-silencing can be described as including two steps. In the first step, dsRNAs introduced into the cell are degraded by a ribonuclease III enzyme, referred to as dicer, into siRNAs of approximately 21 to 23 nucleotides in length that comprise about 19 nucleotide pair duplexes with approximately two nucleotide overhangs at each 3′ end of the siRNA duplex. Hamilton, et al., supra; Berstein, et al., Nature 409:363, 2001; Elbashir, et al., Genes Dev. 15:188, 2001; and Kim, et al., Nature Biotech. 23(2):222, 2005. Dicer has also been implicated in the excision of 21- and 22-nucleotide small temporal RNAs (stRNAs) from precursor RNA of conserved structure that are implicated in translational control. Hutvagner, et al., Science 293:834, 2001.

The second step of dsRNA duplex-mediated targeted gene-silencing involves incorporating the siRNA into a multi-component nuclease complex known as the RNA-induced silencing complex or “RISC.” The RISC identifies mRNA substrates via their homology to the anti-sense strand of the siRNA duplex, and effectuates silencing of gene expression by binding to and destroying the targeted mRNA. Thus, RISC mediates cleavage of single-stranded RNA having sequence complementary to the antisense strand of the siRNA duplex. Cleavage of the target RNA takes place in the middle of the region complementary to the antisense strand of the siRNA duplex. Elbashir, et al., Genes Dev. 15:188, 2001.

For most applications described to date, the nucleotide sequence of the siRNA is selected from conserved regions identified in the target RNA. This approach, however, employs a single RNA target methodology and ignores the possibility that variants of the target RNA (gene variants) may be present or later developed within the cell. Thus, the introduction of an siRNA with a specific nucleotide sequence may target a particular mRNA for destruction, yet remain ineffective in destroying variants of that RNA.

The need to target gene variants is especially crucial when siRNA-mediated gene silencing is used to treat a disease or disorder caused by a virus, whose genes are susceptible to a rapid rate of mutation. In this context, a particular siRNA that targets a specific viral RNA may initially function as a therapeutic agent, but due to the rapid mutation rate of the viral gene, lose the ability to target the viral RNA for degradation. The ability to target gene variants with a single siRNA is also critical where mutant variants of a particular gene associated with a disease state or disorder are present. Finally, gene variant targeting may also be useful where multiple RNAs from a gene family need to be degraded for successful treatment of a patient.

Thus, there remains a long-standing unmet need in the art for compositions and methods that improve the effectiveness of siRNA-mediated gene silencing. In particular, a need exists for siRNAs that effectively reduce the expression of a targeted gene, and variants of that gene that are present within a cell, thereby altering a phenotype or reducing a disease state of the targeted cells.

### SUMMARY OF THE DISCLOSURE

The present disclosure fulfills these and other related needs by providing compositions and methods for increasing the number of target RNAs, such as variants of viral RNAs or endogenous genes, that are susceptible to degradation facilitated by one or more small inhibitory nucleic acid(s) (siRNA(s)). Compositions described herein incorporate one or more universal-binding nucleotide(s) in a first, second, and/or third position in an anti-codon of an anti-sense strand of an siRNA duplex thereby increasing the number of RNA to which the siRNA anti-sense strand specifically binds.

Within certain aspects, the present disclosure provides siRNA, and compositions comprising one or more siRNA, wherein at least one of the siRNA comprise one or more universal-binding nucleotide(s) in the first, second and/or third position in the anti-codon of the anti-sense strand and wherein the siRNA is capable of specifically binding to an RNA, such as an RNA expressed by a target virus. In cases wherein the sequence of the target virus RNA includes one or more single nucleotide substitution, the universal-binding nucleotide comprising siRNA retains its capacity for specifically binding to the target virus RNA thereby mediating gene silencing and, as a consequence, overcoming escape of the target virus to siRNA-mediated gene silencing.

Thus, compositions and methods disclosed herein are useful in reducing the titre of a wide variety of target viruses including, but not limited to, retroviruses, such as human immunodeficiency virus (HIV), as well as respiratory viruses, such as human respiratory syncytial virus, human metapneumovirus, human parainfluenza virus 1, human parainfluenza virus 2, human parainfluenza virus 3, human parainfluenza virus 4a, human parainfluenza virus 4b, influenza A virus, influenza B virus, rhinovirus and influenza C virus.

Non-limiting examples of universal-binding nucleotides that may be suitably employed in the compositions and methods disclosed herein include inosine, 1-β-D-ribofuranosyl-5-nitroindole, and 1-β-D-ribofuranosyl-3-nitropyrrole. For the purpose of the present disclosure, a universal-binding nucleotide is a nucleotide that can form a hydrogen bonded nucleotide pair with more than one nucleotide type.

Non-limiting examples of anti-codons that may be suitably modified within the anti-sense strand of the siRNA duplex include, for example, anti-codons corresponding to the codons for tyrosine (UAU), phenylalanine (UUU or UUC), cysteine (UGU or UGC), histidine (CAU or CAC), asparagine (AAU or AAC), isoleucine (AUA), and aspartic acid (GAU or GAC).

Within certain embodiments, the isoleucine anti-codon UAU, for which AUA is the cognate codon, may be modified such that the third-position uracil (U) nucleotide is substituted with the universal-binding nucleotide inosine (I) to create the anti-codon IAU. Inosine is an exemplary universal-binding nucleotide that can nucleotide-pair with an adenine (A), uracil (U), and cytosine (C) nucleotide, but not with a guanine (G). This modified anti-codon IAU increases the specific-binding capacity of the siRNA molecule and thus permits the siRNA to pair with mRNAs having any one of AUA, UUA, and CUA in the corresponding position of the coding strand thereby expanding the number of available RNA degradation targets to which the siRNA may specifically bind.

Alternatively, the anti-codon AUA may also or alternatively be modified by substituting a universal-binding nucleotide in the second position of the anti-codon such that the anti-codon(s) represented by UIU (second position substitution) or UAI (first position substitution) to generate siRNA that are capable of specifically binding to UAA, UAC AND UAU OR UAU, UCU AND UUU, respectively.

Typically, siRNA of the present disclosure comprise between about 15 base-pairs and about 40 base-pairs; alternatively, between about 18 and about 35 base-pairs or between about 20 and 30 base-pairs such as, for example, either 21, 22, 23, 24, 25, 26, 27, 28, or 29 base-pairs. Within certain embodiments, the siRNA may, optionally, comprise a single-strand 3′ overhang of between 1 nucleotide and 5 nucleotides. Such single-strand 3′ overhangs may be, for example, 1, 2, 3, or 4 nucleotides. Regardless of the precise length of the siRNA duplex and optional overhanging sequence, the siRNA duplex will comprise at least one or more universal-binding nucleotide. Non-limiting examples of universal-binding nucleotides that may be suitably employed in the siRNA of the present disclosure may be selected from the group consisting of inosine, 1-β-D-ribofuranosyl-5-nitroindole, and 1-β-D-ribofuranosyl-3-nitropyrrole.

Typically, siRNA disclosed herein will include between about 1 universal-binding nucleotide and about 10 universal-binding nucleotides. For example, siRNA of the present disclosure may include 1, 2, 3, 4, 5, 6, 7, 8, 9, or 10 universal-binding nucleotides. Within certain aspects, the presently disclosed siRNA may comprise a sense strand that is homologous to a sequence of a target gene and an anti-sense strand that is complementary to a sense strand, with the proviso that at least one nucleotide of the anti-sense strand of the otherwise complementary siRNA duplex is replaced by one or more universal-binding nucleotide.

Within other aspects of the present disclosure are provided methods that employ one or more siRNA, and compositions comprising one or more siRNA, wherein at least one of the siRNA comprise one or more universal-binding nucleotide(s) in the first, second and/or third position in the anti-codon of the anti-sense strand of the siRNA duplex is capable of specifically binding to an RNA, such as an RNA expressed by a target virus.

Within certain embodiments, methods disclosed herein comprise the steps of (a) selecting a target gene, wherein the target gene is a target viral gene, for siRNA-mediated gene silencing; (b) designing and/or synthesizing a suitable siRNA for siRNA gene silencing of the target viral gene, wherein the siRNA comprises one or more universal-binding nucleotide in the anti-sense strand; and (c) administering the siRNA to a cell expressing the target viral gene, wherein the siRNA is capable of specifically binding to the target viral gene thereby reducing its expression level in the cell.

Within alternative embodiments, methods disclosed herein comprise the steps of (a) selecting a target gene for siRNA-mediated gene silencing, wherein the target gene is an endogenous gene wherein the endogenous target gene comprises one or more sequence variation from a corresponding wild-type endogenous gene; (b) designing and/or synthesizing a suitable siRNA for siRNA gene silencing of the endogenous target gene, wherein the siRNA comprises one or more universal-binding nucleotide in the anti-sense strand; and (c) administering the siRNA to a cell expressing the endogenous target gene, wherein the siRNA is capable of specifically binding to the endogenous target gene thereby reducing its expression level in the cell.

It will be understood that methods of the present disclosure do not require a priori knowledge of the nucleotide sequence of every possible gene variant(s) targeted by the universal-binding nucleotide comprising siRNA. Initially, the nucleotide sequence of the siRNA may be selected from a conserved region of the target gene.

Within certain embodiments of the presently disclosed methods, one or more anti-codon(s) within the anti-sense strand of the siRNA molecule is modified by substituting a universal-binding nucleotide for a first position (i.e., the wobble nucleotide position) in the anti-codon(s) of the anti-sense strand. Relying on the wobble hypothesis, the first nucleotide-pair substitution allows the “modified siRNA” antisense strand to specifically bind to mRNA wherein a first nucleotide-pair substitution has occurred, but which substitution does not result in an amino acid change in the corresponding gene product owing to the redundancy of the genetic code.

Within alternative embodiments of the presently disclosed methods, one or more anti-codon(s) within the anti-sense strand of the siRNA molecule is modified by substituting a universal-binding nucleotide for a first and/or second position in the anti-codon(s) of the anti-sense strand. By substituting a universal-binding nucleotide for a first and/or second position, the one or more first and/or second position nucleotide-pair substitution allows the “modified siRNA” molecule to specifically bind to RNA wherein a first and/or a second position nucleotide-pair substitution has occurred, wherein the one or more nucleotide-pair substitution results in an amino acid change in the corresponding gene product.

It will be understood that, regardless of the position at which the one or more universal-binding nucleotide is substituted, the siRNA molecule is capable of binding to a target gene and one or more variant(s) thereof thereby facilitating the degradation of the target gene and/or variant thereof via a RISC complex. Thus, the siRNA of the present disclosure are suitable for introduction into cells to mediate targeted post-transcriptional gene silencing of a target gene and/or variants thereof.

Within still further aspects of the present disclosure are provided methods for selecting modified siRNA molecules that are capable of specifically binding to a wide range of desired gene target variants while being incapable of specifically binding to non-desired gene target variants. The selection process disclosed herein is useful, for example, in eliminating modified siRNAs that are capable of exerting a cytotoxic effect resulting from non-specific binding to, and subsequent degradation of, one or more non-target genes.

Certain embodiments disclosed herein provide methods for selecting one or more modified siRNA molecule(s) that employ the step of predicting the stability of an siRNA duplex. Typically, such a prediction is achieved by employing a theoretical melting curve wherein a higher theoretical melting curve indicates an increase in siRNA duplex stability and a concomitant decrease in cytotoxic effect. Alternatively, stability of an siRNA duplex may be determined empirically by measuring the hydridization of a single modified RNA strand containing one or more universal-binding nucleotide(s) to a complementary target gene within, for example, a polynucleotide array. The melting temperature (i.e., the Tₘ value) for each modified RNA and complementary RNA immobilized on the array can be determined and, from this Tₘ value, the relative stability of the modified RNA pairing with a complementary RNA molecule determined.

Alternative embodiments provide methods for selecting one or more universal-binding nucleotide comprising siRNA, which methods employ “off-target” profiling whereby one or more universal-binding nucleotide comprising siRNA is administered to a cell(s), either in vivo or in vitro, and total mRNA is collected, and used to probe a microarray comprising oligonucleotides having one or more nucleotide sequence from a panel of known genes, including non-target genes. The “off-target” profile of the modified RNA is quantified by determining the number of non-target genes having reduced expression levels in the presence of the universal-binding nucleotide comprising siRNA. The existence of “off target” binding indicates an siRNA that is capable of specifically binding to one or more non-target gene. Ideally, a universal-binding nucleotide comprising siRNA applicable to therapeutic use will exhibit a high Tₘ value while exhibiting little or no “off-target” binding.

Still further embodiments provide methods for selecting one or more potentially efficacious universal-binding nucleotide comprising siRNA. Such methods employ one or more reporter gene construct comprising a constitutive promoter, for example the cytomegalovirus (CMV) or phosphoglycerate kinase (PGK) promoter, operably fused to, and capable of modulating the expression of, one or more reporter gene such as, for example, a luciferase gene, a chloramphenicol (CAT) gene, and/or a β-galactosidase gene, which, in turn, is operably fused in-frame with an oligonucleotide. Oligonucleotides may be between about 15 base-pairs and about 40 base-pairs or between about 19 base-pairs and about 30 base-pairs. Exemplary oligonucleotides are 20, 21, 22, 23, 24, 25, 26, 27, 28, or 29 base-pairs. Such oligonucleotides contain a target sequence for the one or more universal-binding nucleotide comprising siRNA.

Individual reporter gene expression constructs may be co-transfected with one or more universal-binding nucleotide comprising siRNA. The capacity of a given universal-binding nucleotide comprising siRNA to reduce the expression level of each of the contemplated gene variants may be determined by comparing the measured reporter gene activity from cells transfected with and without the modified siRNA.

Each of these aspects of the present disclosure will be better understood by reference to the following detailed description of the invention.

### BRIEF DESCRIPTION OF THE FIGURES AND SEQUENCE IDENTIFIERS

FIG. 1A is a bar graph depicting influenza NP gene knockdown activity obtained for the wild-type vector CM06 and siRNA molecules G1499/112-118.

FIG. 1B is a bar graph depicting influenza NP gene knockdown activity obtained for the variant vector CM02 and siRNA molecules G1499/112-118.

FIG. 2A is a bar graph depicting influenza NP gene knockdown activity obtained for the wild-type vector CM06 and siRNA molecules G1498/101-107.

FIG. 2B is a bar graph depicting influenza NP gene knockdown activity obtained for the variant vector CM02 and siRNA molecules G1498/101-107.

FIG. 2C is a bar graph depicting influenza NP gene knockdown activity obtained for the variant vector CM04 and siRNA molecules G1498/101-107.

SEQ ID NO: 1 is a partial nucleotide sequence (CM01; 5′-GGGUCUUAUUUCUUCGGAGA-3′) of the influenza NP gene to which siRNA molecules G1498/101-107 and G1499/112-118 can specifically bind.

SEQ ID NO: 2 is a partial nucleotide sequence (CM02; 5′-GGAUCUUACUUCUUCGGAGA-3′) of the influenza NP gene to which siRNA molecules G1498/101-107 and G1499/112-118 can specifically bind.

SEQ ID NO: 3 is a partial nucleotide sequence (CM03; 5′-GGAUCUUAUUUUUUCGGAGA-3′) of the influenza NP gene to which siRNA molecules G1498/101-107 and G1499/112-118 can specifically bind.

SEQ ID NO: 4 is a partial nucleotide sequence (CM04; 5′-GGAUCUUAUUUCUUUGGAGA-3′) of the influenza NP gene to which siRNA molecules G1498/101-107 and G1499/112-118 can specifically bind.

SEQ ID NO: 5 is a partial nucleotide sequence (CM05; 5′-GGAUCUUAUUUCUUUCGGGGA-3′) of the influenza NP gene to which siRNA molecules G1498/101-107 and G1499/112-118 can specifically bind.

SEQ ID NO: 6 is a partial nucleotide sequence (CM06; 5′-GGAUCUUAUUUCUUUCGGAGA-3′) of the influenza NP gene to which siRNA molecules G1498/101-107 and G1499/112-118 can specifically bind.

SEQ ID NO: 7 is the nucleotide sequence of the antisense strand of siRNA molecule G1498-101 (5′-CUCCGAAGAAAUAAGAUCC-3′).

SEQ ID NO: 8 is the nucleotide sequence of the antisense strand of siRNA molecule G1498-102 (5′-CUCCGAAGAAAUAAGAICC-3′).

SEQ ID NO: 9 is the nucleotide sequence of the antisense strand of siRNA molecule G1498-103 (5′-CUCCGAAGAAAUAIGAUCC-3′).

SEQ ID NO: 10 is the nucleotide sequence of the antisense strand of siRNA molecule G1498-104 (5′-CUCCGAAGAAIUAAGAUCC-3′).

SEQ ID NO: 11 is the nucleotide sequence of the antisense strand of siRNA molecule G1498-105 (5′-CUCCGAAIAAAUAAGAUCC-3′).

SEQ ID NO: 12 is the nucleotide sequence of the antisense strand of siRNA molecule G1498-106 (5′-CUCCIAAGAAAUAAGAUCC-3′).

SEQ ID NO: 13 is the nucleotide sequence of the antisense strand of siRNA molecule G1498-107 (5′-CICCGAAGAAAUAAGAUCC-3′).

SEQ ID NO: 14 is the nucleotide sequence of the antisense strand of siRNA molecule G1499-112 (5′-UCUCCGAAGAAAUAAGAUC-3′).

SEQ ID NO: 15 is the nucleotide sequence of the antisense strand of siRNA molecule G1499-113 (5′-UCUCCGAAGAAAUAAGAIC-3′).

SEQ ID NO: 16 is the nucleotide sequence of the antisense strand of siRNA molecule G1499-114 (5′-UCUCCGAAGAAAUAIGAUC-3′).

SEQ ID NO: 17 is the nucleotide sequence of the antisense strand of siRNA molecule G1499-115 (5′-UCUCCGAAGAAIUAAGAUC-3′).

SEQ ID NO: 18 is the nucleotide sequence of the antisense strand of siRNA molecule G1499-116 (5′-UCUCCGAAIAAAUAAGAUC-3′).

SEQ ID NO: 19 is the nucleotide sequence of the antisense strand of siRNA molecule G1499-117 (5′-UCUCCIAAGAAAUAAGAUC-3′).

SEQ ID NO: 20 is the nucleotide sequence of the antisense strand of siRNA molecule G1499-118 (5′-UCICCGAAGAAAUAAGAUC-3′).

SEQ ID NO: 21 is the nucleotide sequence of the sense strand of siRNA molecule G1498-101 (5′-GGAUCUUALUCUUUCGGAG-3′).

SEQ ID NO: 22 is the nucleotide sequence of the sense strand of siRNA molecule G1498-102 (5′-GGCUCUUAUUUCUUCGGAG-3′).

SEQ ID NO: 23 is the nucleotide sequence of the sense strand of siRNA molecule G1498-103 (5′-GGAUCCUAUUUCUUUUCGGAG-3′).

SEQ ID NO: 24 is the nucleotide sequence of the sense strand of siRNA molecule G1498-104 (5′-GGAUCUUACUUUCUUCGGAG-3′).

SEQ ID NO: 25 is the nucleotide sequence of the sense strand of siRNA molecule G1498-105 (5′-GGAUCUUAUUUCUUCGGAG-3′).

SEQ ID NO: 26 is the nucleotide sequence of the sense strand of siRNA molecule G1498-106 (5′-GGAUCUUALUCUUUCGGAG-3′).

SEQ ID NO: 27 is the nucleotide sequence of the sense strand of siRNA molecule G1498-107 (5′-GGAUCUUAUUUCUUUCGGCG-3′).

SEQ ID NO: 28 is the nucleotide sequence of the sense strand of siRNA molecule G1499-112 (5′-GAUCUUAUUUCUUCGGAGA-3′).

SEQ ID NO: 29 is the nucleotide sequence of the sense strand of siRNA molecule G1499-113 (5′-GCUCUUAUUUCUUCGGAGA-3′).

SEQ ID NO: 30 is the nucleotide sequence of the sense strand of siRNA molecule G1499-114 (5′-GAUCCUAUUUCUUUCGGAGA-3′).

SEQ ID NO: 31 is the nucleotide sequence of the sense strand of siRNA molecule G1499-115 (5′-GAUCUUACUUCUUCGGAGA-3′).

SEQ ID NO: 32 is the nucleotide sequence of the sense strand of siRNA molecule G1499-116 (5′-GAUCUUAUUUCUUUCGGAGA-3′).

SEQ ID NO: 33 is the nucleotide sequence of the sense strand of siRNA molecule G1499-117 (5′-GAUCUUAUUUCUUCGGAGA-3′).

SEQ ID NO: 34 is the nucleotide sequence of the sense strand of siRNA molecule G1499-118 (5′-GAUCUUAUUUCUUCGGCGA-3′).

### DETAILED DESCRIPTION OF THE DISCLOSURE

The present disclosure is predicated upon the discovery that universal-binding nucleotides may be usefully employed to generate siRNA that exhibit increased capacity for specifically binding to one or more variant(s) of a target gene. Thus, provided herein are compositions and methods for increasing the number of target RNAs, such as viral RNAs, susceptible to degradation facilitated by a single small inhibitory nucleic acid (siRNA). Compositions and methods described herein incorporate one or more universal-binding nucleotide(s) in a first, second, and/or third position in an anti-codon of an anti-sense strand of an siRNA duplex thereby increasing the number of RNA to which the siRNA anti-sense strand specifically binds.

The present disclosure may be best understood in reference to the following non-limiting definitions. All references cited herein, whether infra or supra, are hereby incorporated by reference in their entireties.

### Definitions

As used herein the term “cell” is meant to include both prokaryotic (e.g., bacterial) and eukaryotic (e.g., mammalian or plant) cells. Cells may be of somatic or germ line origin, may be totipotent or pluripotent, and may be dividing or non-dividing. Cells can also be derived from or can comprise a gamete or an embryo, a stem cell, or a fully differentiated cell. Thus, the term “cell” is meant to retain its usual biological meaning and can be present in any organism such as, for example, a bird, a plant, and a mammal, including, for example, a human, a cow, a sheep, an ape, a monkey, a pig, a dog, and a cat. Within certain aspects, the term “cell” refers specifically to mammalian cells, such as human cells, that contain one or more siRNA molecule(s) of the present disclosure.

As used herein, the term “RNA” is meant to include polynucleotide molecules comprising at least one ribonucleotide residue. The term “ribonucleotide” is meant to include nucleotides with a hydroxyl group at the 2′ position of a β-D-ribo-furanose moiety. The term RNA includes, for example, double-stranded RNAs; single-stranded RNAs; and isolated RNAs such as partially purified RNA, essentially pure RNA, synthetic RNA, recombinantly produced RNA, as well as altered RNA that differ from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. As disclosed in detail herein, nucleotides in the siRNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

As used herein, the term “subject” is meant to include any mammalian organism, which is a donor or recipient of explanted cells or the cells themselves. “Subject” also refers to an organism to which the siRNA of the invention can be administered. In one embodiment, a subject is either a human or human cells.

The term “universal-binding nucleotide” as used herein refers to a nucleotide analog that is capable of forming base-pairs with each of the natural DNA/RNA nucleotides with little discrimination between them. Non-limiting examples of universal-binding nucleotides include inosine, 1-β-D-ribofuranosyl-5-nitroindole, and/or 1-β-D-ribofuranosyl-3-nitropyrrole.

By “comprising” is meant including, but not limited to, whatever follows the word “comprising.” Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

### Universal-Binding Nucleotide Comprising siRNA

Within certain aspects, the present disclosure provides siRNA, and compositions comprising one or more siRNA, wherein at least one of the siRNA comprises one or more universal-binding nucleotide(s) in the first, second and/or third position in the anti-codon of the anti-sense strand of the siRNA duplex and wherein said siRNA is capable of specifically binding to a RNA, such as an RNA expressed by a target virus. In cases wherein the sequence of the target virus RNA includes one or more single nucleotide substitution, the universal-binding nucleotide comprising siRNA retains its capacity for specifically binding to the target virus RNA thereby mediating gene silencing and, as a consequence, overcoming escape of the target virus to siRNA-mediated gene silencing.

Compositions and methods disclosed herein are useful in the treatment of a wide variety of target viruses including, but not limited to, a retrovirus, such as human immunodeficiency virus (HIV), as well as respiratory viruses, such as human respiratory syncytial virus, human metapneumovirus, human parainfluenza virus 1, human parainfluenza virus 2, human parainfluenza virus 3, human parainfluenza virus 4a, human parainfluenza virus 4b, influenza A virus, influenza B virus, rhinovirus and influenza C virus.

Non-limiting examples of universal-binding nucleotides that may be suitably employed in the compositions and methods disclosed herein include inosine, 1-β-D-ribofuranosyl-5-nitroindole, and 1-β-D-ribofuranosyl-3-nitropyrrole. For the purpose of the present disclosure, a universal-binding nucleotide is a nucleotide that can form a hydrogen bonded nucleotide pair with more than one nucleotide type.

Non-limiting examples for the above compositions includes modifying the anti-codons for tyrosine (AUA) or phenylalanine (AAA or GAA), cysteine (ACA or GCA), histidine (AUG or GUG), asparagine (AUU or GUU), isoleucine (UAU) and aspartate (AUC or GUC) within the anti-codon of the anti-sense strand of the siRNA molecule.

For example, within certain embodiments, the isoleucine anti-codon UAU, for which AUA is the cognate codon, may be modified such that the third-position uracil (U) nucleotide is substituted with the universal-binding nucleotide inosine (I) to create the anti-codon UAI. Inosine is a universal-binding nucleotide that can nucleotide-pair with an adenine (A), uracil (U), and cytosine (C) nucleotide, but not guanine (G). This modified anti-codon UAI increases the specific-binding capacity of the siRNA molecule and thus permits the siRNA to pair with mRNAs having any one of AUA, UUA, and CUA in the corresponding position of the coding strand thereby expanding the number of available RNA degradation targets to which the siRNA may specifically bind.

Alternatively, the anti-codon AUA may also or alternatively be modified by substituting a universal-binding nucleotide in the third or second position of the anti-codon such that the anti-codon(s) represented by UAI (first position substitution) or UIU (second position substitution) to generate siRNA that are capable of specifically binding to AUA, CUA and UUA and AAA, ACA and AUA.

Typically, siRNA of the present disclosure comprise between about 15 base-pairs and about 40 base-pairs; more typically, between about 18 and 35 base-pairs; still more typically between about 20 and 30 base-pairs; and most typically either 21, 22, 23, 24, 25, 26, 27, 28, or 29 nucleotides and may comprise a single-strand overhang of between 0 nucleotides and 5 nucleotides, most typically, the single-strand 3′ overhang is 1, 2, 3, or 4 nucleotides. Regardless of the precise length of the siRNA duplex and optional overhanging sequence, the siRNA duplex will comprise at least one or more universal-binding nucleotide, wherein the at least one or more universal-binding nucleotide may be selected from the group consisting of inosine, 1-β-D-ribofuranosyl-5-nitroindole, and 1-β-D-ribofuranosyl-3-nitropyrrole.

Typically, siRNA disclosed herein will include between about 1 universal-binding nucleotide and about 10 universal-binding nucleotides. For example, siRNA of the present disclosure may include 1, 2, 3, 4, 5, 6, 7, 8, 9, or 10 universal-binding nucleotides. Within certain aspect, the presently disclosed siRNA may comprise a sense strand that is homologous to a sequence of a target gene and an anti-sense strand that is complementary to the sense strand, with the proviso that at least one nucleotide of the anti-sense strand of the otherwise complementary siRNA duplex is replaced by one or more universal-binding nucleotide.

It will be understood that, regardless of the position at which the one or more universal-binding nucleotide is substituted, the siRNA molecule is capable of binding to a target gene and one or more variant(s) thereof thereby facilitating the degradation of the target gene and/or variant thereof via a RISC complex. Thus, the siRNA of the present disclosure are suitable for introduction into cells to mediate targeted post-transcriptional gene silencing of a target gene and/or variants thereof. When an siRNA is inserted into a cell, the siRNA duplex is then unwound, and the antisense strand of the duplex is loaded into an assembly of proteins to form the RNA-induced silencing complex (RISC).

Within the silencing complex, the siRNA molecule is positioned so that RNAs can bump into it. The RISC will encounter thousands of different RNAs that are in a typical cell at any given moment. But the siRNA of the RISC will adhere well only to an RNA that closely complements its own nucleotide sequence. So unlike an interferon response to a viral infection, the silencing complex is highly selective in choosing its target RNAs.

When a matched RNA finally docks onto the siRNA, an enzyme know as dicer cuts the captured RNA strand in two. The RISC then releases the two pieces of the RNA (now rendered incapable of directing protein synthesis) and moves on. The RISC itself stays intact capable of finding and cleaving another RNA.

One embodiment of the present disclosure is comprised of nanoparticles of double-stranded RNA less than 100 nanometers (nm). More, specifically, the double-stranded RNA is less than about 30 base-pairs in length, preferably 20-25 nucleotide base-pairs in length.

### Synthesis of Universal-Binding Nucleotide Comprising siRNA

Synthesis of nucleic acids greater than 100 nucleotides in length is difficult using automated methods, and the therapeutic cost of such molecules is prohibitive. In the present disclosure, small nucleic acid motifs (“small” refers to nucleic acid motifs no more than 100 nucleotides in length, preferably no more than 80 nucleotides in length, and most preferably no more than 50 nucleotides in length; e.g., individual siRNA oligonucleotide sequences or siRNA sequences synthesized in tandem) are preferably used for exogenous delivery. The simple structure of these molecules increases the ability of the nucleic acid to invade targeted regions of protein and/or RNA structure. Exemplary molecules of the instant invention are chemically synthesized.

Oligonucleotides (e.g., certain modified oligonucleotides comprising one or more universal-binding nucleotide) are synthesized using protocols known in the art, for example as described in Caruthers, et al., Methods in Enzymology 211:3-19, 1992; Thompson, et al., International PCT Publication No. WO 99/54459; Wincott, et al., Nucleic Acids Res. 23:2677-2684, 1995; Wincott, et al., Methods Mol Bio. 74:59, 1997; Brennan, et al., Biotechnol. Bioeng. 61:33-45, 1998; and Brennan, U.S. Pat. No. 6,001,311.

Synthesis of universal-binding nucleotide comprising siRNA molecules of the present disclosure generally follows the procedure described in Usman, et al., J. Am. Chem. Soc. 109:7845, 1987; Scaringe, et al., Nucleic Acids Res. 18:5433, 1990; and Wincott, et al., Nucleic Acids Res. 23:2677-2684, 1995; and Wincott, et al., Methods Mol. Bio. 74:59, 1997.

Alternatively, the universal-binding nucleotide comprising siRNA molecules of the present disclosure can be synthesized separately and joined together post-synthetically, for example, by ligation (Moore, et al., Science 256:9923, 1992; Draper, et al., International PCT Publication No. WO 93/23569; Shabarova, et al., Nucleic Acids Research 19:4247, 1991; Bellon, et al., Nucleosides & Nucleotides 16:951, 1997; Bellon, et al., Bioconjugate Chem. 8:204, 1997, or by hybridization following synthesis and/or deprotection.

### Compositions Comprising Universal-Binding Nucleotide Comprising siRNA

Universal-binding nucleotide comprising siRNA of the present disclosure, either individually or in combination with one or more other compound, can be used to treat diseases or conditions as discussed herein or as otherwise known in the art. To treat a particular disease or condition, the universal-binding nucleotide comprising siRNA molecules can be administered to a patient or can be administered to other appropriate cells evident to those skilled in the art, individually or in combination with one or more compound under conditions suitable for the treatment.

For example, the universal-binding nucleotide comprising siRNA molecules described herein can be used in combination with other known treatments and/or therapeutic agents to treat a wide variety of conditions, particularly viral infections. Non-limiting examples of other therapeutic agents that can be readily combined with a universal-binding nucleotide comprising siRNA molecule of the invention include, for example, enzymatic nucleic acid molecules; allosteric nucleic acid molecules; antisense, decoy, or aptamer nucleic acid molecules; antibodies such as monoclonal antibodies; small molecules; and other organic and/or inorganic compounds including metals, salts and ions.

Thus, the invention features compositions comprising one or more universal-binding nucleotide comprising siRNA molecules of the invention in an acceptable carrier, such as a stabilizer, buffer, and the like. The negatively charged siRNA molecules of the invention may be administered to a patient by any standard means, with or without stabilizers, buffers, and the like, to form a composition suitable for treatment. When it is desired to use a liposome delivery mechanism, standard protocols for formation of liposomes can be followed. The compositions of the present disclosure may also be formulated and used as tablets, capsules or elixirs for oral administration, aerosolizable mixtures for nasal or pulmonary administration, suppositories for rectal administration, sterile solutions, and suspensions for injectable administration, either with or without other compounds known in the art.

The present disclosure also includes pharmaceutically acceptable formulations of the compounds and compositions described herein. These formulations include salts of the above compounds, e.g., acid addition salts such as salts of hydrochloric acid, hydrobromic acid, acetic acid, and benzene sulfonic acid.

A pharmaceutical composition or formulation refers to a composition or formulation in a form suitable for administration, e.g., systemic administration, into a cell or patient such as a human. Suitable forms, in part, depend upon the use or the route of entry, for example oral, intranasal, transdermal, or by injection. Such forms should not prevent the composition or formulation from reaching a target cell (i.e., a cell to which the negatively charged nucleic acid is desirable for delivery). For example, pharmaceutical compositions injected into the blood stream should be soluble. Other factors are known in the art, and include considerations such as toxicity and forms that prevent the composition or formulation from exerting its effect.

### Methods for Selecting Universal-Binding Nucleotide Comprising siRNA

As indicated above, the present disclosure also provides methods for selecting modified siRNA molecules that are capable of specifically binding to a wide range of desired gene target variants while being incapable of specifically binding to non-desired gene target variants. The selection process disclosed herein is useful, for example, in eliminating modified siRNAs that are capable of exerting a cytotoxic effect resulting from non-specific binding to, and subsequent degradation of, one or more non-target gene.

Certain embodiments disclosed herein provide methods for selecting one or more modified siRNA molecule(s) that employ the step of predicting the stability of an siRNA duplex. Typically, such a prediction is achieved by employing a theoretical melting curve wherein a higher theoretical melting curve indicates an increase in siRNA duplex stability and a concomitant decrease in cytotoxic effects. Alternatively, stability of an siRNA duplex may be determined empirically by measuring the hydridization of a single modified RNA strand containing one or more universal-binding nucleotide(s) to a complementary target gene within, for example, a polynucleotide array. The melting temperature (i.e., the Tₘ value) for each modified RNA and complementary RNA immobilized on the array can be determined and, from this Tₘ value, the relative stability of the modified RNA pairing with a complementary RNA molecule determined.

Kawase, et al., have described an analysis of the nucleotide-pairing properties of dI to A, C, G, and T, which was achieved by measuring the hybridization of oligonucleotides (ODNs) with dI in various positions to complementary sets of ODNs made as an array. Nucleic Acids Research 14:7727-7736, 1986. The relative strength of nucleotide-pairing is I-C>I-A>I-G˜I-T. Generally, dI containing duplexes show lower Tm values when compared to the corresponding WC nucleotide pair. The stabilization of dI by pairing was in order of dC>dA>dG>dT>dU. (See, Table 1).

TABLE 1

d(GGAAAAXAAAAGG) (SEQ ID NO: 35)
d(CC TTTT YT T TT CC) (SEQ ID NO: 36)

| Duplex | | | | | |
|---|---|---|---|---|---|
| X/Y nu- | | Corresponding | | Corresponding | |
| cleotide | Tₘ | WT sequence | Tₘ | WT sequence | Tₘ |
| pair | ° C. | where X/Y are | ° C. | where X/Y are | ° C. |
| I/C | 50.9 | G/C | 52.8 | | |
| I/A | 47.0 | T/A | 52.8 | U/A | 51.0 |
| I/G | 43.8 | G/C | 52.8 | | |
| I/T | 43.4 | A/T | 52.8 | A/U | 51.0 |
| I/U | 39.7 | A/U | 51.0 | | |

The following rules, derived from Kawase, et al., are applicable to the design and selection of universal-binding nucleotide comprising siRNA according to the present disclosure, wherein the universal-binding nucleotide is insosine: (a) when XY=IC, Tₘ (A₂₆₀=0.5) is measured to be 51.1° C. while the corresponding wild type double-strand siRNA melts at 59.2° C., an approximately 4° decrease per substitution in the melting temperature; (b) when XY=IA, Tₘ (A₂₆₀=0.5) is measured to be 44.7° C. while the corresponding wild type double-strand siRNA melts at 42.3° C. (that is, replacement of two Ts with dI in the self-complementary duplex shown in Table 2 stabilizes the duplex marginally—˜1.2° C. per substitution); (c) when XY=IG, Tₘ (A₂₆₀=0.5) is measured to be only 35.0° C. while the corresponding wild type double-strand siRNA (XY=CG) melts at 510° C., an approximately 8° C. decrease per substitution in the melting temperature; (d) when XY=IT, the siRNA duplex is not expected to show cooperative melting, but the wild sequence (XY=AT) melts at 54.8° C. (indicating that the I-T nucleotide pair is very unstable—that is, replacement of 2 As in the siRNA duplex with two dIs; (e) incorporation of 4 dI in the duplex presented in Table 2 destabilizes the duplex significantly.

From the thermodynamic values calculated using van't Hoff plots according to a two state model, Kawase, et al., conclude that the sequence of purine-pyrimidine is favored in double strand formation due to nucleotide stacking. For instance the duplex formation of XY=AT is more favored than XY=CG and TA. (See, Table 2)

TABLE 2

Tₘ values of self-complementary duplexes

| | | | | | |
|---|---|---|---|---|---|
| d(GGGAAXYTTCCC) | Tₘ | Tₘ | Tₘ | Tₘ | Tₘ |
| (SEQ ID NO: 37) | (A₂₆₀ = 0.25) | (A₂₆₀ = 0.5) | (A₂₆₀ = 1.0) | (A₂₆₀ = 2.0) | (A₂₆₀ = 3.0) |

| | | | | | |
|---|---|---|---|---|---|
| IC | 48.5 | 51.1 | 52.6 | 55.0 | 55.8 |
| IA | 42.5 | 44.7 | 45.8 | 48 | 49.0 |
| IG | — | 35.0 | 36.5 | 38.3 | 39.7 |
| IT | — | — | — | — | — |
| II | — | — | — | — | — |
| GC | 56.5 | 59.2 | 60.7 | 62.8 | 63.5 |
| GA | 42.0 | 44.1 | 45.9 | 48.5 | 50.3 |
| GG | — | 33.2 | 36.7 | 38.4 | 40.8 |
| GT | — | — | — | — | — |
| AT | 51.6 | 54.8 | 57.0 | 58.0 | 58.8 |
| TA | 40.6 | 42.3 | 43.9 | 45.2 | 45.9 |
| CG | 50.4 | 51.0 | 52.2 | 55.5 | 56.2 |
| AC | — | — | — | — | — |
| CT | — | — | — | — | — |

Note 1: Tₘs were measured at various concentrations and have been shown by their A₂₆₀.
Note 2: Where there is no value, the duplex did not show cooperative melting.

Alternative embodiments provide methods for selecting one or more universal-binding nucleotide comprising siRNA, which methods employ “off-target” profiling whereby one or more universal-binding nucleotide comprising siRNA is administered to a cell(s), either in vivo or in vitro, and total mRNA is collected, and used to probe a microarray comprising oligonucleotides having one or more nucleotide sequence from a panel of known genes, including non-target genes. The “off-target” profile of the modified siRNA is quantified by determining the number of non-target genes having reduced expression levels in the presence of the universal-binding nucleotide comprising siRNA. The existence of “off target” binding indicated an siRNA that is capable of specifically binding to one or more non-target gene. Ideally, a universal-binding nucleotide comprising siRNA applicable to therapeutic use will exhibit a high Tₘ value while exhibiting little or no “off-target” binding.

Still further embodiments provide methods for selecting one or more potentially efficacious universal-binding nucleotide comprising siRNA. Such methods employ one or more reporter gene construct comprising a constitutive promoter, for example the cytomegalovirus (CMV) or phosphoglycerate kinase (PGK) promoter, operably fused to, and capable of modulating the expression of, one or more reporter gene such as, for example, a luciferase gene, a chloramphenicol (CAT) gene, and/or a β-galactosidase gene, which, in turn, is operably fused in-frame with an oligonucleotide (typically between about 15 base-pairs and about 40 base-pairs, more typically between about 19 base-pairs and about 30 base-pairs, most typically 20, 21, 22, 23, 24, 25, 26, 27, 28, or 29 base-pairs) that contains a target sequence for the one or more universal-binding nucleotide comprising siRNA.

Individual reporter gene expression constructs may be co-transfected with one or more universal-binding nucleotide comprising siRNA. The capacity of a given universal-binding nucleotide comprising siRNA to reduce the expression level of each of the contemplated gene variants may be determined by comparing the measured reporter gene activity from cells transfected with and without the modified siRNA.

Within other aspects of the present disclosure are provided methods that employ one or more siRNA, and compositions comprising one or more siRNA, wherein at least one of the siRNA comprise one or more universal-binding nucleotide(s) in the first, second and/or third position in the anti-codon of the anti-sense strand of the siRNA duplex is capable of specifically binding to an mRNA, such as an mRNA expressed by a target virus.

Within certain embodiments, methods disclosed herein comprise the steps of (a) selecting a target gene, wherein the target gene is a target viral gene, for siRNA-mediated gene silencing; (b) designing and/or synthesizing a suitable siRNA for siRNA gene silencing of the target viral gene, wherein the siRNA comprises one or more universal-binding nucleotide in the anti-sense strand; and (c) administering the siRNA to a cell expressing the target virus gene, wherein the siRNA is capable of specifically binding to the target virus gene thereby reducing its expression level in the cell.

Within alternative embodiments, methods disclosed herein comprise the steps of (a) selecting a target gene for siRNA-mediated gene silencing, wherein the target gene is an endogenous gene wherein the endogenous target gene comprises one or more sequence variation from a corresponding wild-type endogenous gene; (b) designing and/or synthesizing a suitable siRNA for siRNA gene silencing of the endogenous target gene, wherein the siRNA comprises one or more universal-binding nucleotide in the anti-sense strand; and (c) administering the siRNA to a cell expressing the endogenous target gene, wherein the siRNA is capable of specifically binding to the endogenous target gene thereby reducing its expression level in the cell.

It will be understood that methods of the present disclosure do not require a priori knowledge of the nucleotide sequence of every possible gene variant(s) targeted by the universal-binding nucleotide comprising siRNA. Initially, the nucleotide sequence of the siRNA is selected from a conserved region of the target gene.

Within certain embodiments of the presently disclosed methods, one or more anti-codon(s) within the anti-sense strand of the siRNA molecule is modified by substituting a universal-binding nucleotide for a first position (i.e., the wobble nucleotide position) in the anti-codon(s) of the anti-sense strand. Relying on the wobble hypothesis, the first nucleotide-pair substitution allows the “modified siRNA” anti-sense strand to specifically bind to RNA wherein a first nucleotide-pair substitution has occurred, but which substitution does not result in an amino acid change in the corresponding gene product owing to the redundancy of the genetic code.

Within alternative embodiments of the presently disclosed methods, one or more anti-codon(s) within the anti-sense strand of the siRNA molecule is modified by substituting a universal-binding nucleotide for a second and/or third position in the anti-codon(s) of the anti-sense strand. By substituting a universal-binding nucleotide for a first and/or second position, the one or more first and/or second position nucleotide-pair substitution allows the “modified siRNA” molecule to specifically bind to mRNA wherein a first and/or a second position nucleotide-pair substitution has occurred, wherein the one or more nucleotide-pair substitution results in an amino acid change in the corresponding gene product.

### Administration of Universal-Binding Nucleotide Comprising siRNA

Methods for the delivery of nucleic acid molecules are described in Akhtar, et al., Trends Cell Bio. 2:139, 1992; “Delivery Strategies for Antisense Oligonucleotide Therapeutics” (ed. Akhtar, 1995); Maurer, et al., Mol. Membr. Biol. 16:129-140, 1999; Hofland and Huang, Handbook Exp. Pharmacol. 137:165-192, 1999; and Lee, et al., ACS Symp. Ser. 752:184-192, 2000. Sullivan, et al., PCT WO 94/02595, further describes the general methods for delivery of enzymatic nucleic acid molecules. These protocols can be utilized for the delivery of virtually any nucleic acid molecule.

As used herein, the term “systemic administration” is meant to include in vivo systemic absorption or accumulation of drugs in the blood stream followed by distribution throughout the entire body. Administration routes which lead to systemic absorption include, without limitation: intravenous, subcutaneous, intraperitoneal, intranasal, inhalation, oral, intrapulmonary and intramuscular. Each of these administration routes expose the desired negatively charged polymers, for example, nucleic acids, to an accessible diseased tissue. The rate of entry of a drug into the circulation has been shown to be a function of molecular weight or size.

Nucleic acid molecules can be administered to cells by a variety of methods known to those of skill in the art, including, but not restricted to, encapsulation in liposomes; by iontophoresis; or by incorporation into other vehicles, such as hydrogels, cyclodextrins, biodegradable nanocapsules, and bioadhesive microspheres; or by proteinaceous vectors (O'Hare and Normand, International PCT Publication No. WO 00/53722). Alternatively, the nucleic acid/vehicle combination may be locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry, et al., Clin. Cancer Res. 5:2330-2337, 1999, and Barry, et al., International PCT Publication No. WO 99/31262.

The universal-binding nucleotide comprising siRNA molecules of the instant invention can be used as pharmaceutical agents. Pharmaceutical agents prevent, modulate the occurrence, or treat (alleviate a symptom to some extent, preferably all of the symptoms) of a disease state in a patient.

As used herein, the phrase “pharmaceutically acceptable formulation” is meant to include compositions or formulations that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, Fundam. Clin. Pharmacol. 13:16-26, 1999); biodegradable polymers, such as poly(DL-lactide-coglycolide) microspheres for sustained release delivery after intracerebral implantation (Emerich, et al., Cell Transplant 8:47-58, 1999, Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry 23:941-949, 1999).

The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

Other non-limiting examples of delivery strategies for the nucleic acid molecules of the instant invention include material described in Boado, et al., J. Pharm. Sci. 87:1308-1315, 1998; Tyler, et al., FEBS Lett. 421:280-284, 1999; Pardridge, et al., PNAS USA 92:5592-5596, 1995; Boado, Adv. Drug Delivery Rev. 15:73-107, 1995; Aldrian-Herrada, et al., Nucleic Acids Res. 26:4910-4916, 1998; and Tyler, et al., PNAS USA. 96:7053-7058, 1999.

The invention also features the use of the composition comprising surface-modified liposomes containing poly(ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug. Lasic, et al., Chem. Rev. 95:2601-2627, 1995; Ishiwata, et al., Chem. Pharm. Bull. 43:1005-1011, 1995. Such liposomes have been shown to accumulate selectively in tumors, presumably by extravasation and capture in the neovascularized target tissues. Lasic, et al., Science 267:1275-1276, 1995; Oku, et al., Biochim. Biophys. Acta 1238:86-90, 1995. The long-circulating liposomes enhance the pharmacokinetics and pharmacodynamics of DNA and RNA, particularly compared to conventional cationic liposomes which are known to accumulate in tissues of the MPS. Liu, et al., J. Biol. Chem. 42:24864-24870, 1995; Choi, et al., International PCT Publication No. WO 96/10391; Ansell, et al., International PCT Publication No. WO 96/10390; and Holland, et al., International PCT Publication No. WO 96/10392. Long-circulating liposomes are also likely to protect drugs from nuclease degradation to a greater extent compared to cationic liposomes, nucleotided on their ability to avoid accumulation in metabolically aggressive MPS tissues such as the liver and spleen.

The present disclosure also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co., A. R. Gennaro ed., 1985. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The present disclosure also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro ed., 1985, hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

The universal-binding nucleotide comprising siRNA molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for delivery by inhalation or spray, especially intranasal delivery, can be prepared according to any method known to the art for the manufacture of pharmaceutical compositions and such compositions can contain one or more carriers, enhancers, and/or preservative agents in order to provide pharmaceutically acceptable preparations. Within the mucosal delivery formulations and methods of the invention, the universal-binding nucleotide comprising siRNA molecule can be combined or coordinately administered with a suitable carrier or vehicle for mucosal delivery. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, can be found in the U.S. Pharmacopeia National Formulary, 1857-1859, 1990. Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of administration.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring, and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The universal-binding nucleotide comprising siRNA molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Universal-binding nucleotide comprising siRNA molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon 5 the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The universal-binding nucleotide comprising siRNA molecules of the present disclosure may also be administered to a patient in combination with other therapeutic compounds to increase the overall therapeutic effect. The use of multiple compounds to treat an indication may increase the beneficial effects while reducing the presence of side effects.

In one embodiment, the inventive compositions suitable for administering universal-binding nucleotide comprising siRNA molecules of the invention to specific cell types, such as hepatocytes. For example, the asialoglycoprotein receptor (ASGPr) is unique to hepatocytes and binds branched galactose-terminal glycoproteins, such as asialoorosomucoid (ASOR). Wu and Wu, J. Biol. Chem. 262:4429-4432, 1987. Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains. Baenziger and Fiete, Cell 22:611-620, 1980, and Connolly, et al., J. Biol. Chem. 257:939-945, 1982. Lee and Lee obtained this high specificity through the use of N-acetyl-D-galactosamine as the carbohydrate moiety, which has higher affinity for the receptor, compared to galactose. Glycoconjugate J. 4: 317-328 (1987). This “clustering effect” has also been described for the binding and uptake of mannosyl-terminating glycoproteins or glycoconjugates. Ponpipom, et al., J. Med. Chem. 24:1388-1395, 1981. The use of galactose and galactosamine nucleotided conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention.

### EXAMPLES

### Example 1

### Stability of Universal-Binding Nucleotide Comprising siRNA in Rat Plasma

This Example discloses a suitable animal model system for determining the in vivo stability of a universal-binding nucleotide comprising siRNA of the present disclosure.

A 20 μg aliquot of each universal-binding nucleotide comprising siRNA duplex of are mixed with 200 μl of fresh rat plasma incubated at 37° C. At various time points (0, 30, 60 and 20 min), 50 μl of the mixture are taken out and immediately extracted by phenol:chloroform. SiRNAs are dried following precipitation by adding 2.5 volumes of isopropanol alcohol and subsequent washing step with 70% ethanol. After dissolving in water and gel loading buffer the samples are analyzed on 20% polyacrylamide gel, containing 7 M urea and visualized by ethidium bromide staining and quantitated by densitometry. The level of degradation at each time point may be assessed by electrophoresis on a PAGE gel.

### Example 2

### Measurement of Gene Knockdown Activity by Universal-Binding Nucleotide Comprising siRNA

This Example demonstrates the utility of siRNA containing the universal-binding nucleotide ribo-inosine (I) at locations within the antisense strand that correspond to sites of sequence variation in the NP gene from a number of influenza isolates.

Each of the six variant influenza virus NP gene sequences was cloned into the psiCHECK™-2 plasmid vector (Promega, Madison, Wis.). The psiCHECK™-2 plasmid vector is designed to provide a quantitative and rapid assessment of RNA interference (RNAi) by monitoring changes in expression of a target gene (i.e., influenza virus NP gene variant CM01-06) fused to a Renilla luciferase reporter gene. The influenza NP gene was cloned into the psiCHECK™-2 plasmid within its multiple cloning region, which is downstream of the Renilla translational stop codon, thereby generating a fusion mRNA. Initiation of the RNAi process by one or more of the GM1498/101-107 or GM1499/112-118 siRNAs towards the influenza virus NP gene results in cleavage and subsequent degradation of the fusion mRNA. Decreases in Renilla luciferase activity correlate with the siRNA's RNAi activity.

For influenza NP-luciferase knockdown experiments, HeLa S3 cells were seeded in a 96 well plate with reduced serum medium (OptiMEM I; Invitrogen, Carlsbad, USA) at 20K cells/100 ul/well and co-transfected in triplicate (with Lipofectamine 2000; Invitrogen, Carlsbad, Calif.) with one of the six (6) influenza NP gene-luciferase reporter vectors (CM01 thru CM06) in combination with one of the fourteen (14) siRNA (selected from G1498-101 thru 107 or G1499-112 thru 118) as indicated in Table 3.

Plasmid vectors and siRNA were diluted in OptiMEM I to a final concentration of 10 nM siRNA and 70 ng plasmid per 25 μl. All transfections were carried out by incubating cells at 37° C. and 5% CO₂ for 3 hours, followed by removing the transfection reagent, replenishing cells with complete media and culturing overnight.

TABLE 3

Partial Sequences of NP Genes and Full
Sequences of siRNA Antisense Strands

| Influenza | | |
|---|---|---|
| NP Gene | Partial Influenza NP Gene | |
| Coding | Coding Sequence used in | |
| Sequence | Luciferase Reporter | Sequence |
| Designation | Construct | Identifier |
| CM01 | 5′-GGGUCUUAUUUCUUCGGAGA-3′ | SEQ ID NO: 1 |
| CM02 | 5′-GGAUCUUACUUCUUCGGAGA-3′ | SEQ ID NO: 2 |
| CM03 | 5′-GGAUCUUAUUUUUUCGGAGA-3′ | SEQ ID NO: 3 |
| CM04 | 5′-GGAUCUUAUUUCUUUGGAGA-3′ | SEQ ID NO: 4 |
| CM05 | 5′-GGAUCUUAUUUCUUCGGGGA-3′ | SEQ ID NO: 5 |
| CM06 | 5′-GGAUCUUAUUUCUUCGGAGA-3′ | SEQ ID NO: 6 |
| siRNA | | |
| Antisense | | |
| Strand | siRNA Antisense Strand | Sequence |
| Designation | Sequence | Identifier |
| G1498-101- | 5′-CUCCGAAGAAAUAAGAUCC-3′ | SEQ ID NO: 7 |
| AS | | |
| G1498-102- | 5′-CUCCGAAGAAAUAAGAICC-3′ | SEQ ID NO: 8 |
| AS | | |
| G1498-103- | 5′-CUCCGAAGAAAUAIGAUCC-3′ | SEQ ID NO: 9 |
| AS | | |
| G1498-104- | 5′-CUCCGAAGAAIUAAGAUCC-3′ | SEQ ID NO: 10 |
| AS | | |
| G1498-105- | 5′-CUCCGAAIAAAUAAGAUCC-3′ | SEQ ID NO: 11 |
| AS | | |
| G1498-106- | 5′-CUCCIAAGAAAUAAGAUCC-3′ | SEQ ID NO: 12 |
| AS | | |
| G1498-107- | 5′-CICCGAAGAAAUAAGAUCC-3′ | SEQ ID NO: 13 |
| AS | | |
| siRNA Sense | | |
| Strand | siRNA Sense Strand | Sequence |
| Designation | Sequence | Identifier |
| G1498/101-S | 5′-GGAUCUUAUUUCUUCGGAG-3′ | SEQ ID NO: 21 |
| G1498/102-S | 5′-GGCUCUUAUUUCUUCGGAG-3′ | SEQ ID NO: 22 |
| G1498/103-S | 5′-GGAUCCUAUUUCUUCGGAG-3′ | SEQ ID NO: 23 |
| G1498/104-S | 5′-GGAUCUUACUUCUUCGGAG-3′ | SEQ ID NO: 24 |
| G1498/105-S | 5′-GGAUCUUAUUUCUUCGGAG-3′ | SEQ ID NO: 25 |
| G1498/106-S | 5′-GGAUCUUAUUUCUUCGGAG-3′ | SEQ ID NO: 26 |
| G1498/107-S | 5′-GGAUCUUAUUUCUUCGGCG-3′ | SEQ ID NO: 27 |
| siRNA | | |
| Antisense | | |
| Strand | siRNA Antisense Strand | Sequence |
| Designation | Sequence | Identifier |
| G1499-112- | 5′-UCUCCGAAGAAAUAAGAUC-3′ | SEQ ID NO: 14 |
| AS | | |
| G1499-113- | 5′-UCUCCGAAGAAAUAAGAIC-3′ | SEQ ID NO: 15 |
| AS | | |
| G1499-114- | 5′-UCUCCGAAGAAAUAIGAUC-3′ | SEQ ID NO: 16 |
| AS | | |
| G1499-115- | 5′-UCUCCGAAGAAIUAAGAUC-3′ | SEQ ID NO: 17 |
| AS | | |
| G1499-116- | 5′-UCUCCGAAIAAAUAAGAUC-3′ | SEQ ID NO: 18 |
| AS | | |
| G1499-117- | 5′-UCUCCIAAGAAAUAAGAUC-3′ | SEQ ID NO: 19 |
| AS | | |
| G1499-118- | 5′-UCICCGAAGAAAUAAGAUC-3′ | SEQ ID NO: 20 |
| AS | | |
| siRNA Sense | | |
| Strand | siRNA Sense Strand | Sequence |
| Designation | Sequence | Identifier |
| G1499/112-S | 5′-GAUCUUAUUUCUUCGGAGA-3′ | SEQ ID NO: 28 |
| G1499/113-S | 5′-GCUCUUAUUUCUUCGGAGA-3′ | SEQ ID NO: 29 |
| G1499/114-S | 5′-GAUCCUAUUUCUUCGGAGA-3′ | SEQ ID NO: 30 |
| G1499/115-S | 5′-GAUCUUACUUCUUCGGAGA-3′ | SEQ ID NO: 31 |
| G1499/116-S | 5′-GAUCUUAUUUCUUCGGAGA-3′ | SEQ ID NO: 32 |
| G1499/117-S | 5′-GAUCUUAUUUCUUCGGAGA-3′ | SEQ ID NO: 33 |
| G1499/118-S | 5′-GAUCUUAUUUCUUCGGCGA-3′ | SEQ ID NO: 34 |

Luciferase activity expressed from the reporter vector was detected with the Promega E2940 Dual-Glo Luciferase Assay System. Light emission was detected with a Perkin Elmer Wallac Victor3 1420 multilabel counter. Table 4 summarizes the NP-luciferase knockdown activity for each siRNA expressed as a percentage. Qneg represents a random, non-specific siRNA molecule that functioned as the negative control. The observed Qneg knockdown activity was normalized to 100% (100% gene expression levels) and the knockdown activity for each siRNA was presented as a relative percentage of the negative control.

TABLE 4

| | siRNA 1498 | | siRNA 1499 | |
|---|---|---|---|---|
| | Vector/ | | Vector/ | |
| Pairing | siRNA | % Reduction | siRNA | % Reduction |
| Wild-type | CM06/101 | 77% | CM06/112 | 80% |
| I:G vs U:G | CM01/102 | 59% vs 75% | CM01/113 | 21% vs 47% |
| I:C vs A:C | CM02/104 | 77% vs 54% | CM02/115 | 81% vs 2% |
| I:U vs G:U | CM03/105 | 49% vs 70% | CM03/116 | 11% vs 3% |
| I:U vs G:U | CM04/106 | 72% vs 67% | CM04/117 | 0% vs 16% |
| I:G vs U:G | CM05/107 | 66% vs 71% | CM05/118 | 0% vs 0% |

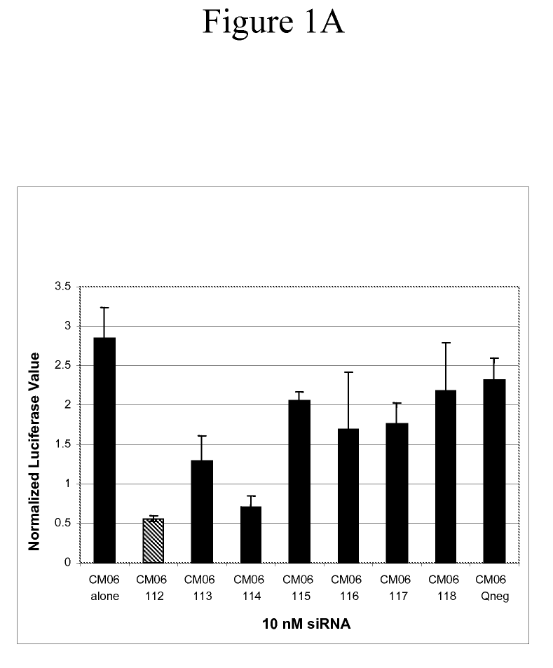

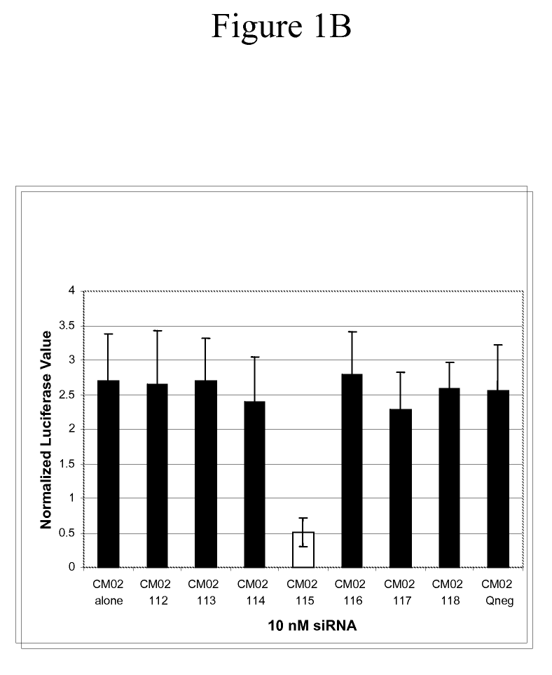

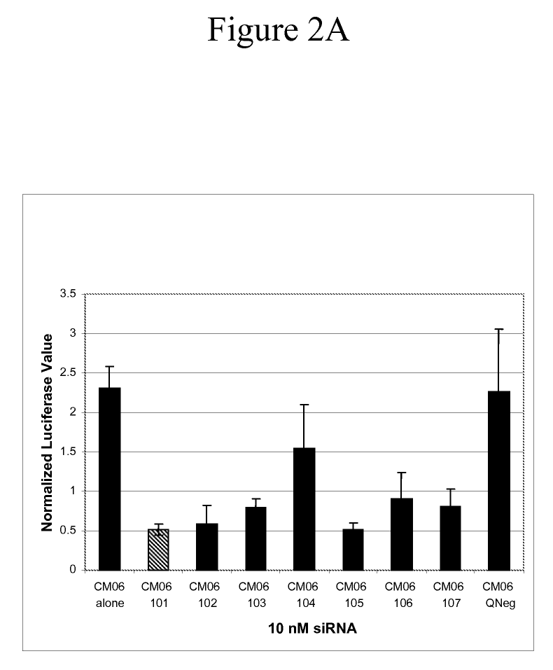

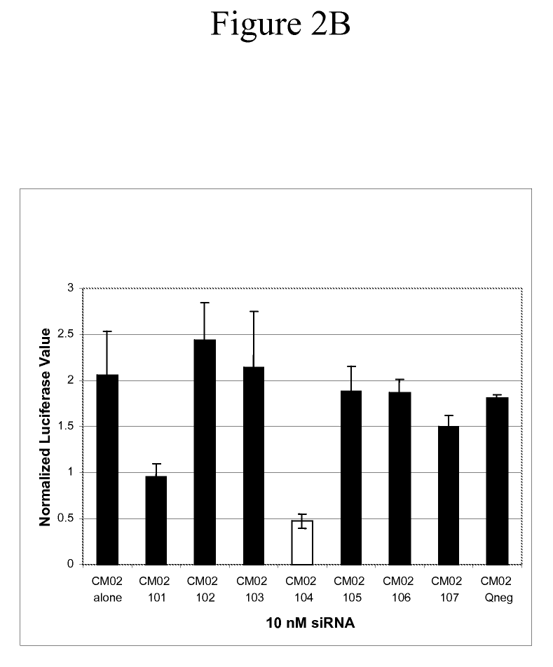

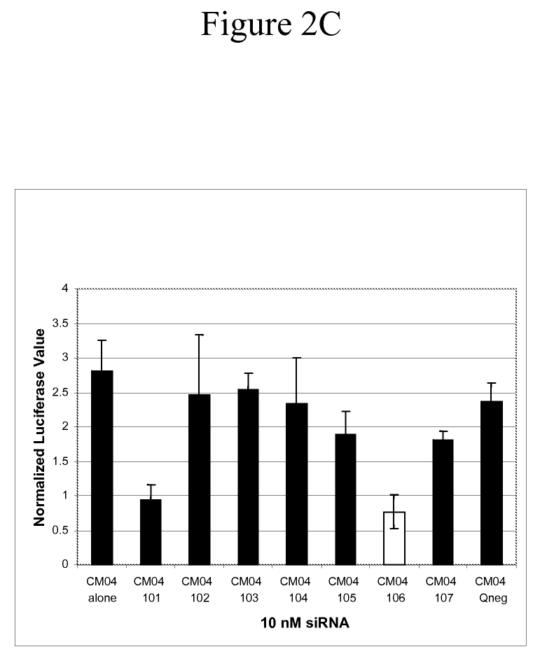

FIGS. 1A-B (siRNA 1499), FIGS. 2A-C (siRNA 1498) and Table 4 disclose the luciferase reduction results. Each bar graph of FIGS. 1A-B and FIGS. 2A-C corresponds to one vector sequence (CM01-CM06) and the individual bars within each bar graph are data obtained for the unsubstituted (G1499/112 and G1498/101) and single ribo-I substituted (G1499/113-118 and G1498/102-107) siRNA molecules. Controls are plasmid alone, first bar each graph, and QNeg siRNA cotransfection, last bar each graph. FIGS. 1A and 2A are data obtained for the wild-type vector CM06 and siRNA G1499/112-118 and G1498/101-107 siRNA molecules, respectively. FIG. 1A shows 80% reduction with CM06 and wild-type (112) and significant reduction compared to Qneg with 113 and 114. FIG. 2A shows 77% reduction with CM06 and wild-type (101) and greater than 50% reduction with 102, 103, 105, 106, and 107. Modest reductions were observed for the variant vector CM01 and siRNA G1499/112-118; however, only 112 showed a reduction greater than Qneg. The CM01 and G1498/101-107 siRNA molecules all showed greater reduction than Qneg with 101 and 102 having the greatest reductions (more than 50%). FIGS. 1B and 2B are data obtained for the variant vector CM02 and siRNA G1499/112-118 and G1498/101-107 siRNA molecules, respectively. FIG. 1B shows at least 80% reduction for CM02 and 115. FIG. 2B shows at least 80% reduction for CM02 and 104. The CM03 and siRNA G1499/112-118 molecules showed modest reductions (greater than Qneg) with 112, 116, and 117; and the CM03 and G1498/101-107 siRNA molecules showed reductions greater than Qneg with 103 and 105. FIG. 2E are data obtained for the variant vector CM04 and siRNA G1498/101-107 siRNA molecules. FIG. 2C showed reduction greater than 60% with 104 or 106. CM04 and siRNA G1499/122-118 showed reductions slightly less than Qneg with 112, 114, 115, and 116. The data obtained for the variant vector CM05 and siRNA G1499/112-118 showed no significant reductions compared to Qneg, and data for vector GM05 and G1498/101-107 siRNA molecules showed significant reductions compared to Qneg for 101, 102, 105, 106, and 107; the reductions for 101, 105, and 107 were greater than 50%.

In each Figure, the hatched bar are data obtained for the no-ribo-I siRNA against its perfect-match target. Bars with siRNAs 102-107 correspond to ribo I substitutions pairing with one vector sequence per graph. Light colored bars indicate ribo I substitutions placed to accommodate a mismatch between the vector influenza sequence and G1499 siRNA or G1498 siRNA.

These data demonstrate that single I:C pairing, with no second mismatch between the siRNA and vector influenza sequence resulted in activity comparable to perfect pairing. Other ribo-inosine pairs resulted in reduced activity as compared to either perfect pairing or a ribo-ribo mismatch. In total, the data presented herein demonstrate that siRNA comprising at least one universal-binding nucleotide are capable of enhancing the ability of the siRNA to downregulate expression of one or more target genes as exemplified by downregulation of the expression of the NP gene of influenza virus.

### Example 3

### Measurement of Off Target Effect by Universal-Binding Nucleotide Comprising siRNA

This Example provides a suitable methodology for measuring off-target effects mediated by universal-binding nucleotide comprising siRNA of the present disclosure.

Although siRNA of the present disclosure may be suitably employed for disrupting the expression of variant target genes, there remains the possibility that such siRNA may affect the expression of one or more non-target gene(s). Thus, an off-target profile may be generated for siRNAs that target a variant of an otherwise wild-type gene, such as a viral gene or an endogenous gene. Agilent microarrays may be employed that consist of 60-mer probe oligonucleotide targets representing, for example, 18,500 well-characterized, full-length human genes.

It is expected that siRNA modifications will have a significant effect on reducing off-target responses. The extent of G:U nucleotide pairing in all the identified siRNA off-target interactions are evaluated and, therefore, the potential of universal-binding nucleotides to eliminate the off-target effects by the suppression of G:U wobble may be ascertained.

The teachings of all of references cited herein including patents, patent applications and journal articles are incorporated herein in their entirety by reference. Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. It is noted, however, that the various publications discussed herein are incorporated solely for their disclosure prior to the filing date of the present application, and the inventors reserve the right to antedate such disclosure by virtue of prior invention.

What is claimed is:

1. A small inhibitory nucleic acid (siRNA) molecule comprising between about 15 base-pairs and about 40 base-pairs and at least one universal-binding nucleotide.

2. The siRNA molecule of claim 1 wherein said universal-binding nucleotide is selected from the group consisting of inosine, 1-β-D-ribofuranosyl-5-nitroindole, and 1-β-D-ribofuranosyl-3-nitropyrrole.

3. The siRNA molecule of claim 2 wherein said siRNA comprises a sense strand that is homologous to a sequence of a target gene and an anti-sense strand that is complementary to said sense strand, and wherein at least one nucleotide of the siRNA anti-sense strand sequence is replaced by a universal-binding nucleotide selected from the group consisting of inosine, 1-β-D-ribofuranosyl-5-nitroindole, and 1-β-D-ribofuranosyl-3-nitropyrrole.

4. The siRNA molecule of claim 3 wherein said siRNA molecule comprises a double-stranded region.

5. The siRNA molecule of claim 4, wherein the siRNA molecule further comprises a 3′-overhang.

6. The siRNA molecule of claim 4 wherein at least one 5′ terminal ribonucleotide of the anti-sense strand of the double stranded region of said siRNA is replaced by a universal-binding nucleotide.

7. The siRNA molecule of claim 4 wherein at least two 5′ terminal ribonucleotides of the anti-sense strand of the double stranded region of the siRNA sequence are replaced by a universal-binding nucleotide.

8. The siRNA molecule of claim 4 wherein at least one 5′ terminal ribonucleotide of the anti-sense strand of the double stranded region of the siRNA sequence is replaced by a universal-binding nucleotide selected from the group consisting of inosine, 1-β-D-ribofuranosyl-5-nitroindole, and 1-β-D-ribofuranosyl-3-nitropyrrole.

9. The siRNA molecule of claim 4 wherein at least two 5′ terminal ribonucleotides of the anti-sense strand of the double stranded region of the siRNA sequence are replaced by a universal-binding nucleotide selected from the group consisting of inosine, 1-β-D-ribofuranosyl-5-nitroindole, and 1-β-D-ribofuranosyl-3-nitropyrrole.

10. The siRNA molecule of claim 4 wherein at least one uradine ribonucleotide of the anti-sense stand of the double stranded region of the siRNA sequence is replaced by a universal-binding nucleotide selected from the group consisting of inosine, 1-β-D-ribofuranosyl-5-nitroindole, and 1-β-D-ribofuranosyl-3-nitropyrrole.

11. The siRNA molecule of claim 4 wherein at least two uradine ribonucleotides of the anti-sense strand of the double stranded region of the siRNA sequence are replaced by a universal-binding nucleotide selected from the group consisting of inosine, 1-β-D-ribofuranosyl-5-nitroindole, and 1-β-D-ribofuranosyl-3-nitropyrrole.

12. The siRNA molecule of claim 4 wherein at least three uradine ribonucleotides of the anti-sense strand of the double stranded region of the siRNA sequence are replaced by a universal-binding nucleotide selected from the group consisting of inosine, 1-β-D-ribofuranosyl-5-nitroindole, and 1-β-D-ribofuranosyl-3-nitropyrrole.

13. The siRNA molecule of claim 4, wherein all of the uradine ribonucleotides of the anti-sense strand of the double stranded region of the siRNA sequence are replaced by a universal-binding nucleotide selected from the group consisting of inosine, 1-β-D-ribofuranosyl-5-nitroindole, and 1-β-D-ribofuranosyl-3-nitropyrrole.

14. The siRNA molecule of any one of claims 1-13 wherein said universal-binding nucleotide increases the binding specificity of said siRNA for a target gene when the siRNA is contacted with a biological sample.

15. The siRNA molecule of any one of claims 1-13 wherein said universal-binding nucleotide reduces off-target effects of the siRNA molecule when the siRNA is contacted with a biological cell.

16. The siRNA molecule of claim 1 wherein said siRNA is capable of specifically binding to a variant of a target gene expressed in a virus selected from the group consisting of a retrovirus and a respiratory virus.

17. The siRNA of claim 16 wherein said retrovirus is the human immunodeficiency virus (HIV).

18. The siRNA of claim 16 wherein said respiratory viruses is selected from the group consisting of human respiratory syncytial virus, human metapneumovirus, human parainfluenza virus 1, human parainfluenza virus 2, human parainfluenza virus 3, human parainfluenza virus 4a, human parainfluenza virus 4b, influenza A virus, influenza B virus, rhinovirus and influenza C virus.

19. A method for improving the binding specificity of a double stranded siRNA molecule for a variant of a target gene when said siRNA is contacted with a biological sample, said method comprising the step of preparing an siRNA molecule of any one of claims 1-13.

20. A method for reducing off-target effects of a double stranded siRNA molecule, by preparing an siRNA molecule of any one of claims 1-13.

21. A method reducing the expression of a target viral gene, said method comprising the steps of:
(a) selecting a target gene, wherein the target gene is a target viral gene, for siRNA-mediated gene silencing;
(b) designing and/or synthesizing a suitable siRNA for siRNA gene silencing of the target viral gene, wherein the siRNA comprises one or more universal-binding nucleotide in the anti-sense strand; and
(c) administering the siRNA to a cell expressing the target viral gene, wherein the siRNA is capable of specifically binding to the target viral gene thereby reducing its expression level in the cell.

22. A method reducing the expression of a target endogenous gene, said method comprising the steps of:
(a) selecting a target gene for siRNA-mediated gene silencing, wherein the target gene is an endogenous gene wherein the endogenous target gene comprises one or more sequence variation from a corresponding wild-type endogenous gene;
(b) designing and/or synthesizing a suitable siRNA for siRNA gene silencing of the endogenous target gene, wherein the siRNA comprises one or more universal-binding nucleotide in the anti-sense strand; and
(c) administering the siRNA to a cell expressing the endogenous target gene, wherein the siRNA is capable of specifically binding to the endogenous target gene thereby reducing its expression level in the cell.


SEQUENCE LISTING (37 sequences)

SEQ ID NO: 1 - RNA, 20 nt, Artificial Sequence
  Description of Artificial Sequence Synthetic oligonucleotide
gggucuuauuucuucggaga

SEQ ID NO: 2 - RNA, 20 nt, Artificial Sequence
  Description of Artificial Sequence Synthetic oligonucleotide
ggaucuuacuucuucggaga

SEQ ID NO: 3 - RNA, 20 nt, Artificial Sequence
  Description of Artificial Sequence Synthetic oligonucleotide
ggaucuuauuuuuucggaga

SEQ ID NO: 4 - RNA, 20 nt, Artificial Sequence
  Description of Artificial Sequence Synthetic oligonucleotide
ggaucuuauuucuuuggaga

SEQ ID NO: 5 - RNA, 20 nt, Artificial Sequence
  Description of Artificial Sequence Synthetic oligonucleotide
ggaucuuauuucuucgggga

SEQ ID NO: 6 - RNA, 20 nt, Artificial Sequence
  Description of Artificial Sequence Synthetic oligonucleotide
ggaucuuauuucuucggaga

SEQ ID NO: 7 - RNA, 19 nt, Artificial Sequence
  Description of Artificial Sequence Synthetic oligonucleotide
cuccgaagaaauaagaucc

SEQ ID NO: 8 - RNA, 19 nt, Artificial Sequence
  Description of Artificial Sequence Synthetic oligonucleotide
cuccgaagaaauaagancc

SEQ ID NO: 9 - RNA, 19 nt, Artificial Sequence
  Description of Artificial Sequence Synthetic oligonucleotide
cuccgaagaaauangaucc

SEQ ID NO: 10 - RNA, 19 nt, Artificial Sequence
  Description of Artificial Sequence Synthetic oligonucleotide
cuccgaagaanuaagaucc

SEQ ID NO: 11 - RNA, 19 nt, Artificial Sequence
  Description of Artificial Sequence Synthetic oligonucleotide
cuccgaanaaauaagaucc

SEQ ID NO: 12 - RNA, 19 nt, Artificial Sequence
  Description of Artificial Sequence Synthetic oligonucleotide
cuccnaagaaauaagaucc

SEQ ID NO: 13 - RNA, 19 nt, Artificial Sequence
  Description of Artificial Sequence Synthetic oligonucleotide
cnccgaagaaauaagaucc

SEQ ID NO: 14 - RNA, 19 nt, Artificial Sequence
  Description of Artificial Sequence Synthetic oligonucleotide
ucuccgaagaaauaagauc

SEQ ID NO: 15 - RNA, 19 nt, Artificial Sequence
  Description of Artificial Sequence Synthetic oligonucleotide
ucuccgaagaaauaaganc

SEQ ID NO: 16 - RNA, 19 nt, Artificial Sequence
  Description of Artificial Sequence Synthetic oligonucleotide
ucuccgaagaaauangauc

SEQ ID NO: 17 - RNA, 19 nt, Artificial Sequence
  Description of Artificial Sequence Synthetic oligonucleotide
ucuccgaagaanuaagauc

SEQ ID NO: 18 - RNA, 19 nt, Artificial Sequence
  Description of Artificial Sequence Synthetic oligonucleotide
ucuccgaanaaauaagauc

SEQ ID NO: 19 - RNA, 19 nt, Artificial Sequence
  Description of Artificial Sequence Synthetic oligonucleotide
ucuccnaagaaauaagauc

SEQ ID NO: 20 - RNA, 19 nt, Artificial Sequence
  Description of Artificial Sequence Synthetic oligonucleotide
ucnccgaagaaauaagauc

SEQ ID NO: 21 - RNA, 19 nt, Artificial Sequence
  Description of Artificial Sequence Synthetic oligonucleotide
ggaucuuauuucuucggag

SEQ ID NO: 22 - RNA, 19 nt, Artificial Sequence
  Description of Artificial Sequence Synthetic oligonucleotide
ggcucuuauuucuucggag

SEQ ID NO: 23 - RNA, 19 nt, Artificial Sequence
  Description of Artificial Sequence Synthetic oligonucleotide
ggauccuauuucuucggag

SEQ ID NO: 24 - RNA, 19 nt, Artificial Sequence
  Description of Artificial Sequence Synthetic oligonucleotide
ggaucuuacuucuucggag

SEQ ID NO: 25 - RNA, 19 nt, Artificial Sequence
  Description of Artificial Sequence Synthetic oligonucleotide
ggaucuuauuucuucggag

SEQ ID NO: 26 - RNA, 19 nt, Artificial Sequence
  Description of Artificial Sequence Synthetic oligonucleotide
ggaucuuauuucuucggag

SEQ ID NO: 27 - RNA, 19 nt, Artificial Sequence
  Description of Artificial Sequence Synthetic oligonucleotide
ggaucuuauuucuucggcg

SEQ ID NO: 28 - RNA, 19 nt, Artificial Sequence
  Description of Artificial Sequence Synthetic oligonucleotide
gaucuuauuucuucggaga

SEQ ID NO: 29 - RNA, 19 nt, Artificial Sequence
  Description of Artificial Sequence Synthetic oligonucleotide
gcucuuauuucuucggaga

SEQ ID NO: 30 - RNA, 19 nt, Artificial Sequence
  Description of Artificial Sequence Synthetic oligonucleotide
gauccuauuucuucggaga

SEQ ID NO: 31 - RNA, 19 nt, Artificial Sequence
  Description of Artificial Sequence Synthetic oligonucleotide
gaucuuacuucuucggaga

SEQ ID NO: 32 - RNA, 19 nt, Artificial Sequence
  Description of Artificial Sequence Synthetic oligonucleotide
gaucuuauuucuucggaga

SEQ ID NO: 33 - RNA, 19 nt, Artificial Sequence
  Description of Artificial Sequence Synthetic oligonucleotide
gaucuuauuucuucggaga

SEQ ID NO: 34 - RNA, 19 nt, Artificial Sequence
  Description of Artificial Sequence Synthetic oligonucleotide
gaucuuauuucuucggcga

SEQ ID NO: 35 - DNA, 13 bp, Artificial Sequence
  Description of Artificial Sequence Synthetic oligonucleotide
ggaaaanaaaagg

SEQ ID NO: 36 - DNA, 13 bp, Artificial Sequence
  Description of Combined DNA/RNA Molecule Synthetic oligonucleotide
ccttttnttttcc

SEQ ID NO: 37 - DNA, 12 bp, Artificial Sequence
  Description of Artificial Sequence Synthetic oligonucleotide
gggaannttccc